(19) United States
(12) Patent Application Publication
(10) Pub. No.: US 2012/0172822 A1
(43) Pub. Date: Jul. 5, 2012

(54) Foley Catheter Collection System

(75) Inventor: Thomas H. Gilman, Spring Grove, IL (US)

(73) Assignee: HOLLISTER INCORPORATED, Libertyville, IL (US)

(21) Appl. No.: 13/411,383

(22) Filed: Mar. 2, 2012

Related U.S. Application Data
(63) Continuation-in-part of application No. PCT/US2010/047841, filed on Sep. 3, 2010.
(60) Provisional application No. 61/239,607, filed on Sep. 3, 2009.

(51) Int. Cl. A61F 5/44

(52) U.S. Cl. 604/328

(57) ABSTRACT

A Foley catheter drainage collection system which is capable of permitting the removal and reconnection of drainage bags without compromising the sterility of the drainage system lumen. The collection system has a tubing element which includes a drainage valve associated with an end thereof remote from the insertion end of a Foley catheter. The drainage valve associated with the tubing element is movable from an open catheter drainage position to a closed position. The collection system also includes a drainage bag having a first connector element associated therewith and a second connector element is associated with the drainage valve. The second connector element is adapted for mating engagement with the first connector element. The collection system further includes a retaining element for maintaining the connector elements in mating engagement when the drainage valve is disposed in the open catheter drainage position.

### REFERENCE TO RELATED APPLICATIONS

This is a continuation-in-part under 35 USC §120 of International Patent Application No. PCT/US2010/047841, filed Sep. 3, 2010, which claims the benefit of the filing date of U.S. Provisional Appl. No. 61/239,607, filed Sep. 3, 2009. The disclosures of International Patent Application No. PCT/US2010/047841 and U.S. Provisional Appl. No. 61/239,607 are incorporated herein by reference.

### FIELD OF THE DISCLOSURE

The present disclosure is generally directed to a catheter and drainage collection system suitable for use in the drainage of urine from the bladder of a patient and, more particularly, to a Foley (indwelling) catheter collection system that reliably maintains the sterility of the collection system lumen.

### BACKGROUND OF THE DISCLOSURE

Medical professionals are very aware that infections secondary to the use of Foley catheters represent a significant healthcare burden. Clinical research indicates that with current catheter drainage systems perhaps 35% of such infections are due to bacteria entering the drainage system lumen. Once the bacteria have entered the lumen of the drainage system, they can easily move up the lumen of the catheter and into the bladder of the patient.

Because of the recurring problem of bacteria entering the drainage system lumen, there has been a long unmet and important need for a drainage system that can protect the sterility of the lumen. This problem is known to be particularly prevalent during the emptying of urine from the drainage bag. However, it is also a problem to prevent bacteria from entering the drainage system lumen during transfer of the patient when it may be desirable to have the drainage bag disconnected from the indwelling catheter.

Currently available drainage systems for Foley catheters typically include a drainage tubing portion that is simply connected to a drain hub of the Foley catheter and a drainage bag that has a drainage outlet that can be opened and closed. These current systems seek to prevent the ingress of bacteria from the outside environment into the drainage system lumen and then ultimately into the bladder. While these current systems are normally closed against bacterial contamination, they do permit the ingress of bacteria in two distinct and important circumstances that are routinely encountered in the use of indwelling catheters.

In particular, bacterial contamination often occurs when the connection between the Foley catheter and the drainage tubing is broken and when the drainage bag is drained of urine to allow refilling. In each of these situations, the normally closed drainage system for the Foley catheter becomes open to the external environment making it possible for bacteria to enter the system. Nevertheless, highly trained, highly focused nursing care can protect the drainage system from contamination in these situations by ensuring contamination is not introduced even though the system is opened.

Unfortunately, such highly trained, highly focused nursing care is not available in most patient care settings. As a result, it is quite common for the lumen of a Foley catheter drainage system to become contaminated. Once this occurs, it is likely the bacteria will reach the bladder, and the patient will develop a urinary tract infection.

### SUMMARY OF THE DISCLOSURE

Accordingly, the present disclosure is directed to a Foley catheter collection system which ensures sterility of the drainage lumen during removal and reconnection of drainage bags. The collection system comprises a tubing element having an end remote from the insertion end of the Foley catheter which includes a drainage valve movable from an open catheter drainage position to a closed position for preventing the flow of urine from the catheter. The collection system also includes a drainage bag having a first connector element associated with the drainage bag and a second connector element associated with the drainage valve.

More specifically, the second connector element is adapted for mating sealed engagement with the first connector element, and the collection system further includes a retaining element for maintaining the connector elements in mating sealed engagement when the drainage valve is disposed in the open catheter drainage position.

The system is designed so that disengagement of the connector elements requires or results in movement of a sealing element in the second connector element from a first position to a second position and where the second position results in the drainage system lumen being sealed.

Furthermore, the second connector element is designed so that the surfaces that receive the first connector element in mating sealed engagement are easily available for sterilization, e.g. by wiping with a sterilizing fluid or by immersion in a sterilizing fluid.

The system is intended to be easy to use in a way that ensures sterility of the drainage system lumen. To achieve this end, the drainage bag does not have a conventional drain valve. Instead, it is designed to be inexpensive and disposable after a single use. In this regard, it would have either no drain valve, with for example a tear feature to aid emptying, or else at most an inexpensive, single use drain valve.

When a drainage bag is full and in need of emptying, the first step is to disengage the connector elements which, as described above, necessarily results in the system drainage lumen being closed. The disconnected bag can now be taken to a toilet and emptied. The used bag is discarded. A new sterilized bag having a first connector element with a sterile mating surface is prepared for use. As noted above, the mating surface of the second connector element is easily sterilized, e.g., by wiping it with or immersing it in a sterilizing fluid. The first and second connector elements are then engaged and the sealing element is moved to a position which opens the drainage valve, allowing communication between the drainage tubing and the collection bag again. The system is now again closed, all parts of the drainage lumen remain sterile, and urine can again drain freely into the new collection bag.

In one embodiment, the tubing element comprises the catheter tube of a Foley catheter wherein the end of the catheter tube remote from the insertion end is provided with a drainage valve. In another embodiment, the Foley catheter collection system includes use of a conventional Foley catheter having a drain hub. In this embodiment, the tubing element is a separate tube having a first end for permanent connection to the drain hub and a second end remote therefrom which includes a drainage valve.

When the tubing element is a separate tube, the first end of the tubing element may include a barbed connector which is adapted to be inserted into the drain hub of the catheter. The tubing element also may advantageously have a lumen extending from the first end thereof to the drainage valve for carrying urine away from the catheter. Further, the drainage valve may in one embodiment be a rotary valve which is adapted for rotation from an open catheter drainage position to a closed position for preventing the flow of urine from the catheter. Alternatively, the drainage valve may be a spring loaded sealing element valve.

With regard to the rotary valve construction, the valve preferably has a lumen in alignment with the lumen of the tubing element in the open catheter drainage position and out of alignment with the lumen of the tubing element in the closed position. In addition, the second connector element suitably includes a tubular segment disposed in alignment with the lumen of the tubing element when the collection system is in a fully assembled condition. Preferably, the lumen of the rotary valve is also in alignment with the lumen of the tubular segment of the second connector element in the open catheter drainage position and out of alignment therewith when the rotary valve is in the closed position.

Further, the retaining element is preferably associated with the rotary valve for rotation from a position in engagement with the first connector element to a position out of engagement therewith. More specifically, the retaining element is in engagement with the first connector element in the open catheter drainage position and out of engagement with the first connector element in the closed position. Preferably, the first connector element includes a tubular segment which is adapted for coaxially aligned telescopic engagement with the tubular segment of the second connector element.

In order to facilitate proper relative orientation of the first and second connector elements for the retaining element to be able to rotate into and out of engagement with the first connector element, the tubular segments may have an elliptical cross section, and the first and second connector elements can additionally have cooperating slots and protrusions, respectively.

With regard to the alternative spring loaded valve, preferably a spring-loaded sealing element is movable between an open position and a sealed position. In the open position, the spring-loaded sealing element is engaged by an axial finger-like protrusion part of a first connector element associated with a collection bag, thereby holding the spring-loaded valve open when the spring-loaded valve is connected to the collection bag. In the sealed position. When the connector portion of the collection bag is disengaged from the spring-loaded sealing element, a spring urges the spring-loaded sealing element to sealingly engage a sealing surface on a valve housing, such that a flat external surface is provided that is easily sterilized, e.g., by wiping it with or immersing it in a sterilizing fluid.

Still other advantages and features of the present disclosure will become apparent from a consideration of the following specification taken in conjunction with the accompanying drawings.

### BRIEF DESCRIPTION OF THE DRAWINGS

FIG. 1 is a plan view of a collection system for a Foley catheter having a drain hub in accordance with the present disclosure;

FIG. 1A is a plan view of a Foley catheter collection system in accordance with an alternative embodiment of the present disclosure;

FIG. 2A is an exploded perspective view of a drainage valve, connector elements and retaining element for the system of FIG. 1;

FIG. 2B is another exploded perspective view from a different angle of the various components illustrated in FIG. 2A;

FIG. 2C is another exploded perspective view from a different angle of the various components illustrated in FIG. 2A;

FIG. 3 is a perspective view showing the connector elements when assembled and held in place by the retaining element;

FIG. 4 is a perspective view showing the drainage bag and the first connector element apart from the remainder of the collection system;

FIG. 4A is a perspective view identical to FIG. 4 showing a peelable lid covering the opening in the first connector element;

FIG. 5 is a cross-sectional view of an alternate embodiment of a drainage valve of the present disclosure, illustrating the valve in an open condition;

FIG. 6 is a cross-sectional view of a portion of the drainage valve of FIG. 5, illustrating the valve in a closed condition; and

FIG. 7 is a perspective view of the drainage valve of FIG. 5.

### DETAILED DESCRIPTION OF THE PREFERRED EMBODIMENTS

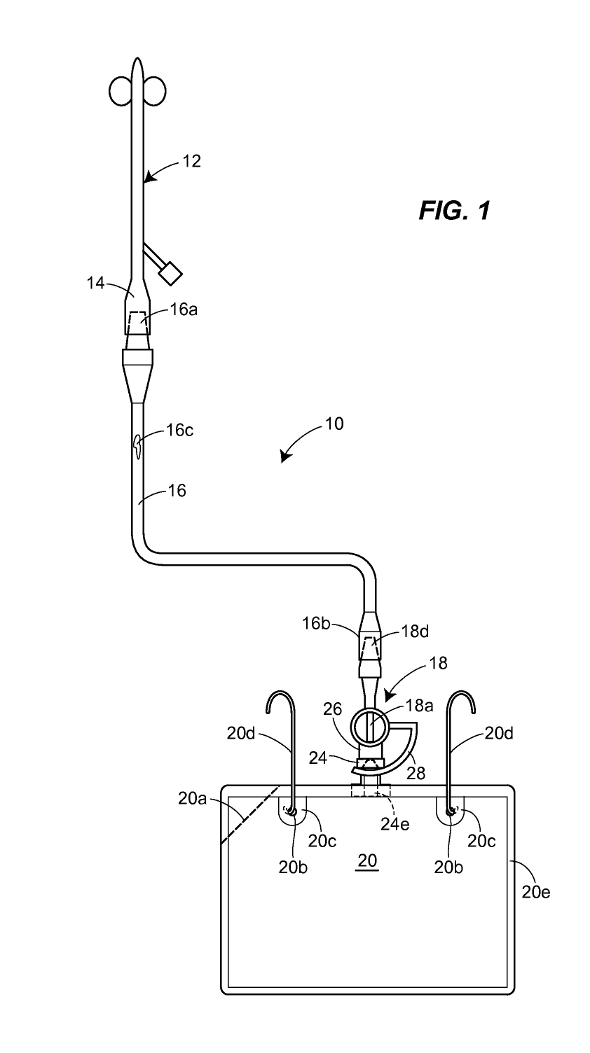

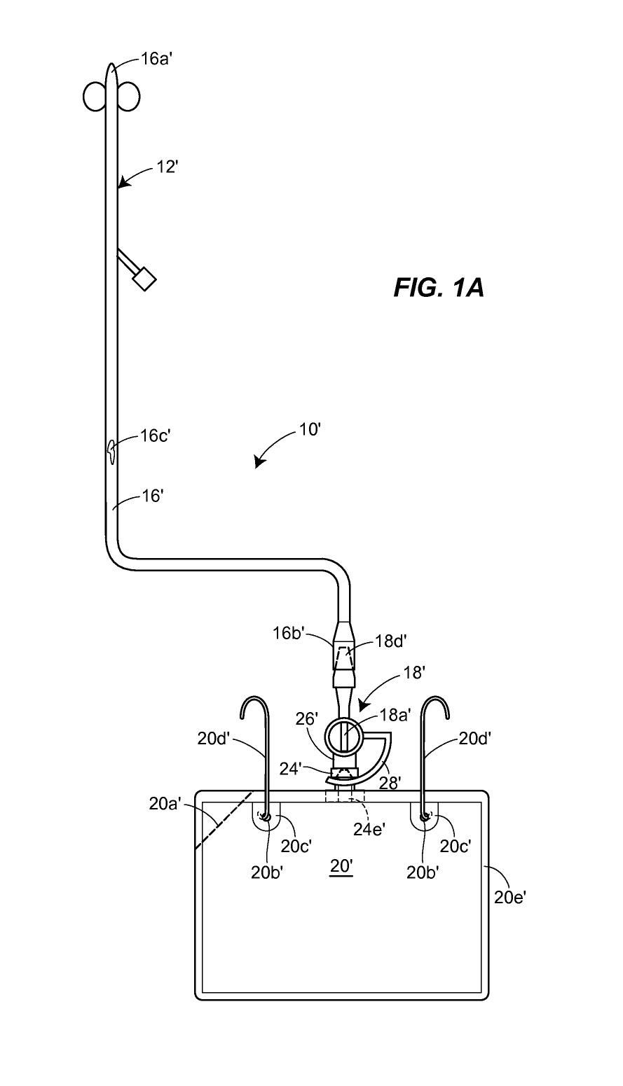

In the illustrations given, and with reference first to FIG. 1, the reference numeral 10 designates generally a Foley catheter collection system in accordance with the present disclosure. The collection system 10 illustrated in FIG. 1 includes a tubing element 16 having a first end 16a for permanent connection to a drain hub 14 of the conventional Foley catheter 12 illustrated in FIG. 1. Further, the tubing element 16 has a second end 16b remote from the first end 16a, and the second end 16b has a drainage valve 18 movable from an open catheter drainage position to a closed position for preventing the flow of urine from the catheter.

In an alternative embodiment illustrated in FIG. 1A, the tubing element 16′ of the Foley catheter collection system 10′ comprises the catheter tube of a specially configured Foley catheter 12′. It will be seen from FIG. 1A that the first end 16a′ of the tubing element 16′ comprises the insertion end of the specially configured Foley catheter 12′ whereas the second end 16b′ of the tubing element 16′ located remote from the insertion end 16a′ is provided with a drainage valve 18′. Thus, the Foley catheter collection systems 10 and 10′ can either be used with a conventional Foley catheter 12 (FIG. 1) or can comprise a specially configured Foley catheter 12′ (FIG. 1A).

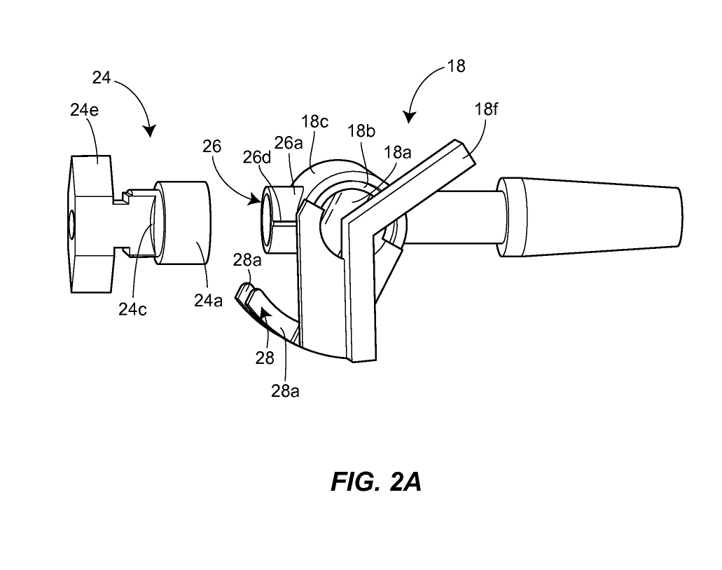

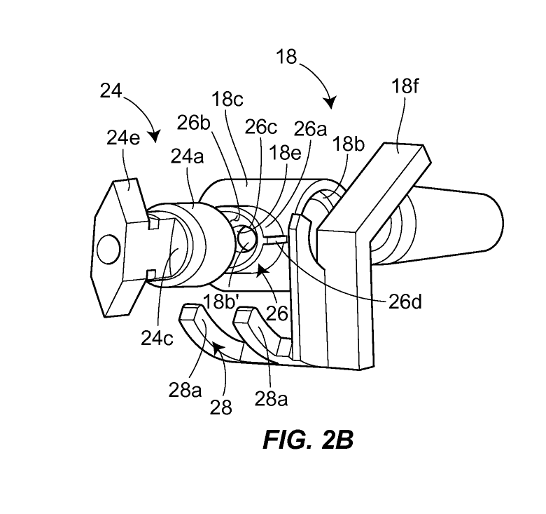

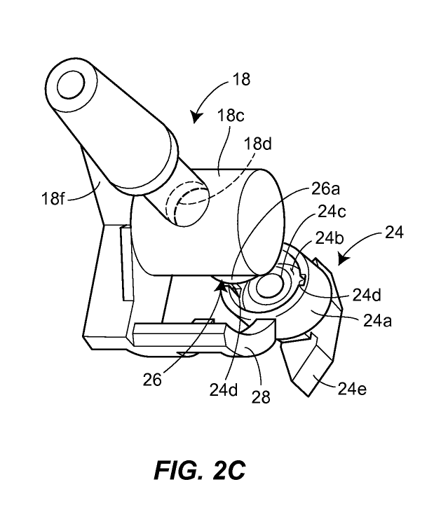

Referring again to FIG. 1, the collection system 10 also includes a drainage bag 20 defining a closed urine collection chamber. The drainage bag 20 has a first connector element 24 associated therewith in fluid communication with the closed urine collection chamber, and the system 10 also includes a second connector element 26 associated with the drainage valve 18 for mating sealed engagement with the first connector element 24. In addition, a retaining element 28 is associated with the rotary valve 18 as best shown in FIGS. 2A-2C.

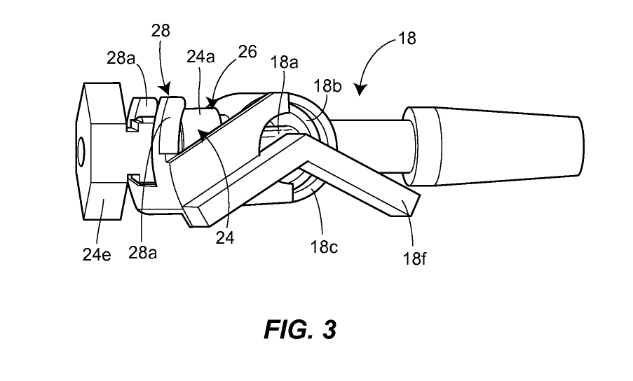

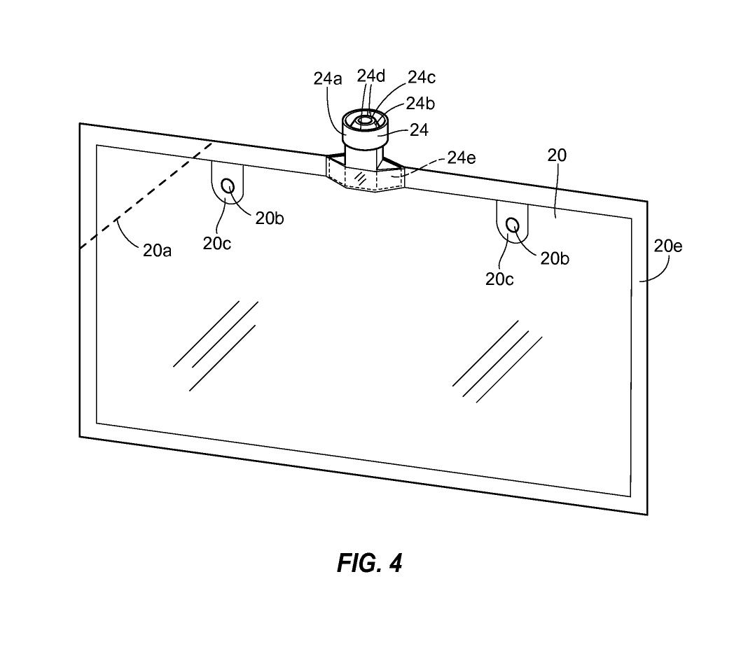

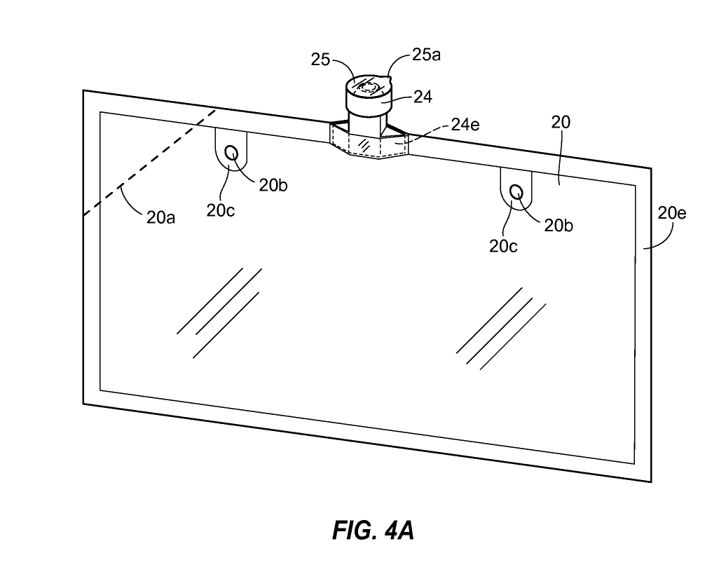

Comparing FIGS. 2A-2C and FIG. 3, it will be seen that the retaining element 28 serves to maintain the connector elements 24 and 26 in mating sealed engagement when the drainage valve 18 is disposed in the open catheter drainage position (FIG. 3). The retaining element 28 can be rotated from a position in which it is in engagement with the first connector element 24 (FIG. 3) to a position in which it is out of engagement therewith (FIGS. 2A-2C). As will be appreciated from FIGS. 2A-2C, the first connector element 24 includes a tubular segment 24a that is preferably elliptical in cross section, adapted for coaxially aligned telescopic engagement with a tubular segment 26a of the second connector element 26, where the tubular segment is again preferably elliptical in cross section.

Inside the tubular segment 24a is a sloping internal engagement surface 24b having an opening 24c of reduced diameter relative to the diameter of the tubular segment 24a. Similarly, there is a corresponding sloping internal engagement surface 26b inside the tubular segment 26a having an opening 26c of reduced diameter relative to the diameter of the tubular segment 26a. These sloping internal engagement surfaces 24b and 26b inside the tubular segments 24b and 26b engage each other and form a liquid tight seal when the retaining element 28 is rotated into a position where it is in engagement with the first connector element 24. In addition, there is preferably an elastomeric material that is shape recoverable on the sloping internal engagement surface 26b to ensure a liquid tight seal each time a new drainage bag 20 is attached.

By utilizing tubular segments 24a and 26a that are elliptical in cross section, it is possible to ensure the proper orientation of the retaining element 28 so it can be rotated into engagement with the first connector element 24 as shown in FIG. 3. The retaining element 28 includes two slightly curved parallel fingers 28a that cooperate with slightly curved surfaces 24c on opposite sides of the first connector element 24 behind the tubular segment 24a to maintain the first and second connector elements 24 and 26 in engagement during use of the collection system 10. Thus, it will be appreciated that the curved fingers 28a are able to slide by and engage the slightly curved surfaces 24c behind the tubular segment 24a to maintain the first and second connector elements 24 and 26 in locked engagement.

To further ensure proper alignment of the first and second connector elements 24 and 26, the first connector element 24 can be provided with diametrically opposed slots 24d inside the tubular segment 24a that can receive corresponding diametrically opposed protrusions 26d outside the tubular segment 26a, so there can be no relative rotation of the first and second connector elements 24 and 26 after they are brought into coaxially aligned telescopic engagement but before the retaining element 28 is rotated into engagement with the first connector element 24. This ensures that when the retaining element 28 is rotated into engagement with the first connector element 24 it will properly engage the surfaces 24c.

Referring specifically to the embodiment of FIG. 1, the first end 16a of the tubing element 16 may include a barbed connector adapted to be inserted into the drain hub 14 of the Foley catheter 12. It will also be appreciated that the separate tube comprising the tubing element 16 has a lumen 16c which the cutaway region illustrates. In conventional fashion, the lumen 16c extends from the barbed connector 16a through the tubing element 16 to the drainage valve 18 to carry urine away from the Foley catheter 12. Inside the tubular segment 24a is a sloping internal engagement surface 24b, and similarly there is a sloping internal engagement surface 26b inside the tubular segment 26a. These sloping internal engagement surfaces engage and form a liquid tight seal when the retaining element 28 is in engagement with the first connector element 24.

With regard to the drainage valve 18, it may advantageously comprise a rotary valve adapted for rotation from an open catheter drainage position (FIGS. 1 and 3) to a closed position for preventing the flow of urine from the catheter (FIGS. 2A-2C). The rotary valve 18 has a lumen defined by a tubular passageway 18a in alignment for communication with the lumen 16c of the tubing element 16 in the open catheter drainage position (FIG. 1). Referring specifically to FIG. 2A, the lumen defined by the tubular passageway 18a is out of alignment with the lumen 16c of the tubing element 16 in the closed position of the drainage valve 18.

When the collection system 10 is assembled, the lumen defined by the tubular segment 26a of the second connector element 26 will be disposed in alignment with the lumen 16c of the tubing element 16. In the open catheter drainage position (FIGS. 1 and 3), the lumen defined by the tubular passageway 18a of the rotary valve 18 is aligned for communication with both the lumen 16c of the tubing element 16 and the lumen inside the tubular segment 26a of the second connector element 26, and in the closed position (FIGS. 2A-2C), the lumen defined by the tubular passageway 18a is out of alignment with the lumen 16c of the tubing element 16 and the lumen which is inside the tubular segment 26a of the second connector element 26. By comparing FIGS. 1 and 3 with FIGS. 2A-2C, it will be seen how the tubular passageway 18a of the rotary valve 18 is able to rotate from the open catheter drainage position to the closed position for preventing the flow of urine from the catheter.

Specifically, the tubular passageway 18a of the drainage valve 18 is able to rotate from the open catheter drainage position shown in FIGS. 1 and 3 to the closed position shown in FIGS. 2A-2C by rotating the retaining element 28 from a position in engagement with the first connector element 24 to a position out of engagement with the first connector element 24. For this to be able to occur, and referring to FIG. 2A, the retaining element 28 is integrally associated with an inner cylindrical valve member 18b of the rotary valve 18 through which the tubular passageway 18a is formed and which can rotate within and relative to the outer cylindrical valve member 18c. As a result, the rotation of the retaining element 28 from the engaged position (FIGS. 1 and 3) to the disengaged position (FIGS. 2A-2C) causes the inner cylindrical valve member 18b to rotate within and relative to the outer cylindrical valve member 18c which causes the tubular passageway 18a to rotate from the open catheter drainage position (FIGS. 1 and 3) to the closed position (FIG. 2A-2C).

As will be seen from FIG. 2C, the outer cylindrical valve member 18c will have an opening 18d which is in direct fluid communication with the lumen 16c of the tubing element 16 and an opening 18e. The opening 18e has the same diameter as and is contiguous with the opening 26c, as shown in FIG. 2B, inasmuch as the cylindrical valve member 18c and the second connector element 26 are preferably integrally formed. Since the opening 18e has the same diameter as and is contiguous with the opening 26c, the opening 18e is in direct fluid communication with the lumen defined by the opening 26c inside the second connector element 26. The openings 18d and 18e in the outer cylindrical valve member 18c are disposed in axial alignment. Further, the openings 18d and 18e in the outer cylindrical valve member 18c will align for direct fluid communication with the openings at the opposite ends of the tubular passageway 18a when the rotary valve 18 is in the open catheter drainage position shown in FIGS. 1 and 3.

In the closed position (FIGS. 2A-2C), the tubular passageway 18a defined by the inner cylindrical valve member 18b is not only out of alignment and, thus, out of communication with the lumen 16c of the tubing element 16, but it is also out of alignment and, thus, out of communication with the lumen inside the tubular segment 26a of the second connector element 26. This occurs because the inner cylindrical valve member 18b through which the tubular passageway 18a is formed will have rotated relative to the outer cylindrical valve member 18c into a position where the inner cylindrical valve member 18b closes off and seals the openings 18d and 18e at opposite ends of the outer cylindrical valve member 18c.

As shown in FIGS. 2A-2C and 3, a valve operating handle 18f can be provided to operate the drainage valve 18, and it is suitably integral with the retaining element 28, and with the inner cylindrical valve member 18b. Rotating the inner cylinder 18b by turning the valve handle 18f rotates the retaining element 28 from the engaged to the disengaged position and causes the tubular passageway 18a to rotate from the open catheter drainage position to the closed position for preventing the flow of urine from the catheter.

As a result, the tubular passageway 18a is not exposed to any bacteria present in the external environment when the rotary valve 18 is in the closed position because the opposite ends of the tubular passageway 18a are closed off and sealed by the outer cylindrical valve member 18c. Moreover, the opening 18e in the outer cylindrical valve member 18c, the sloping internal engagement surface 26b inside the second connector element 26, and the exposed outer surface portion 18b′ of the inner cylindrical valve member 18b are exposed to the environment, but these exposed areas are all positioned where they are easily accessible for sterilization. This is easily accomplished for example by use of a liquid sterilizing agent. The liquid sterilizing agent can be applied in any desired manner, e.g., it can be applied by using a wetted swab or, alternatively, it can be applied by simply immersing the exposed surfaces in the sterilizing liquid just before a new, sterile drainage bag 20 is connected for resumed use of the collection system 10.

From the foregoing, it will be appreciated that the collection system 10 comprises a tubing element 16, a drainage valve 18, and a drainage bag 20 together with associated elements to prevent infections secondary to use of a Foley catheter 12 where the infections are of the type that result from bacteria ascending the lumen of the catheter to reach the bladder. This is accomplished by providing: i) a permanent connection of the tubing element 16 to the drain hub 14 of the Foley catheter 12, ii) a drainage valve 18 and first and second connector elements 24 and 26, and iii) drainage bags 20 that are designed for single use and are only removable in a manner that prevents the ingress of bacteria into the collection system 10.

As previously discussed, the tubing element 16 is preferably designed to have a barbed connector on the end 16a of the tubing element 16 that can be inserted into the drain hub 14 of the Foley catheter 12 to thereby permanently connect the tubing element 16 to the drain hub 14. The other end of the tubing element 16 is preferably permanently connected to the drainage valve 18, and the drainage valve 18 is either permanently connected to, or preferably integral with, the second connector element 26 (see FIGS. 2A, 2B and 3). Thus, the collection system 10 when fully assembled is such that the drainage valve 18 closes off and seals the lumen 16c and, thus, the lumen of the Foley catheter 12 when it is moved from an open catheter drainage position to a closed position to remove the drainage bag 20.

The drainage bag 20 is designed to be very inexpensive so it can be used only a single time and then discarded, e.g., it can be formed of two rectangular sheets of inexpensive plastic such as PVC or polyethylene. These sheets can be sealed together about their perimeters with the end 24e of the first connector element 24 sealed therein. Moreover, the drainage bag 20 is designed so it must be discarded after it has been used, and to this end it does not have a conventional drain spout that can be used for multiple episodes of draining the bag.

For this purpose, the drainage bag 20 may be provided with an inexpensive feature to aid in emptying the bag in the form of a tear line 20a. The tear line 20a is preferably provided at a corner so it can be used to tear off a portion of the bag to form a drainage spout. Once the corner has been torn off to drain the drainage bag 20, it can no longer be used but must be discarded and replaced with a new, sterile drainage bag.

As an additional feature, the drainage bag 20 may have openings 20b through heat seals as at 20c to receive bag hanger hooks 20d. The drainage bag 20 may also have a peripheral seal 20e, and the openings 20b through the heat seals 20c will be understood to facilitate hanging the bag in proximity to a patient using a Foley catheter 12 with a connected collection system 10. The openings 20b, and optionally other openings, can be used for handling the bag during urine drainage and bag disposal.

As another additional feature, a sterile barrier lid 25 having a gripping tab 25a may be peelably sealed to the perimeter surface of the first connector element 24 to maintain sterility inside of new drainage bags. This will also maintain sterility within the drainage lumen of the first connector element 24 without the need to keep the sterilized bags in a separate sterile barrier package. As a result, this will allow, for example, multiple sterilized drainage bags to be provided in a single package with one Foley catheter, without the need for the multiple drainage bags to have their own separate, individual sterile barrier packages.

Referring again to the embodiment of FIG. 1A, the prime reference numerals are provided to indicate the same components as are indicated by the corresponding non-prime reference numerals for the embodiment of FIG. 1. Thus, the foregoing detailed description of the various structural components as well as the operation of the Foley catheter collection system 10 of FIG. 1 will be understood to apply to the Foley catheter collection system 10′. The only difference between the two embodiments is providing a tubing element 16′ comprising a catheter tube for a specially configured Foley catheter 12′ in place of a separate tube 16 for connection to a conventional Foley catheter 12.

It is also possible to provide a drainage valve 18 located at the first end 16a of the tubing element 16 to be directly associated with the drain hub 14 in addition to, or instead of, the drainage valve 18 located at the second end 16b of the tubing element 16. This would provide the capability to safely disconnect the tubing element 16 from the drainage valve 18 and, thus, from the Foley catheter 12, for example, if necessary to move the patient. With this arrangement, the drainage valve 18 associated with the drain hub 14 ensures that the Foley catheter 12 will not become open to the external environment which would make it possible for bacteria to enter the system. Then, the second connector element 26 associated with the drainage valve 18, which remains associated with the drain hub 14 of the Foley catheter 12, can be sterilized and then connected to a new, sterile first connector element such as 24 associated with a new, sterile tubing element 16 and a new sterile drainage bag 20.

With regard to the tubing elements 16 and drainage bags 20 in this embodiment a second drainage valve 18 and associated second connector element 26 may be provided on the end of the tubing element 16 remote from the Foley catheter 12. The second drainage valve 18 can be provided for connecting the end of the tubing element 16 remote from the Foley catheter 12 to a first connector element 24 associated with the drainage bag 20 in the manner described in detail above.

However, it will also be understood that the end of the tubing element 16 remote from the Foley catheter 12 can be made integral with drainage bag 20, if desired, to simplify the system and potentially reduce costs.

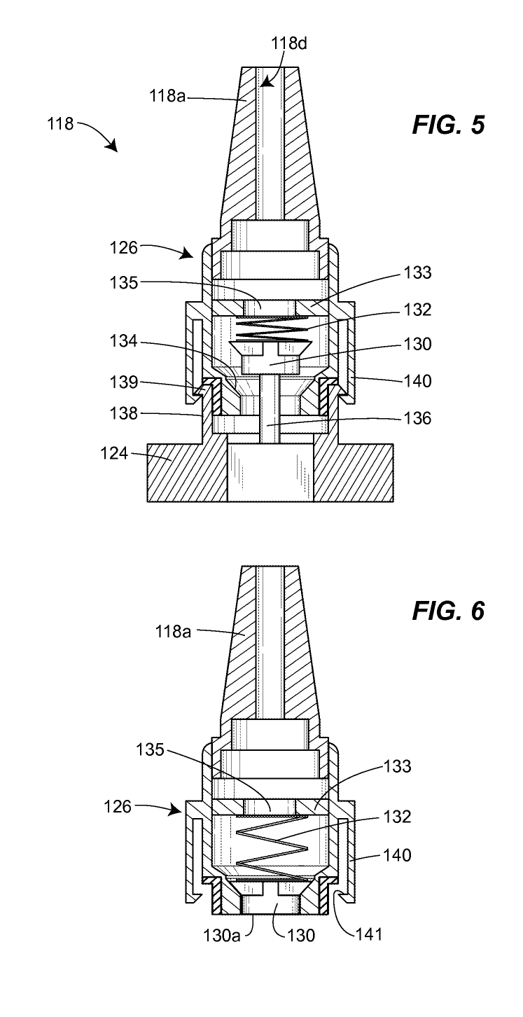

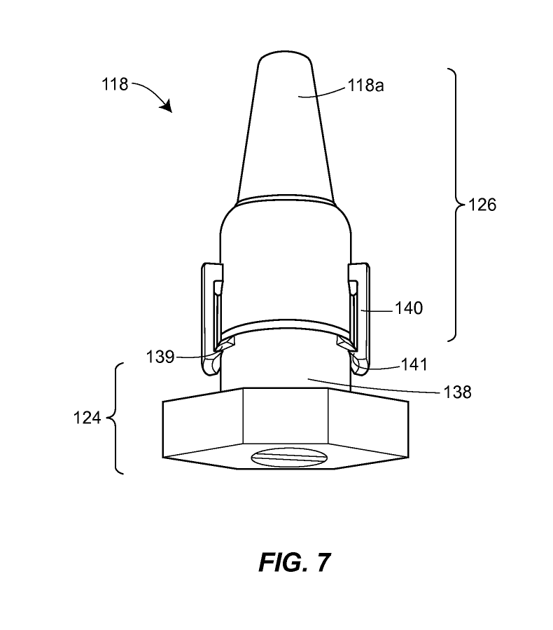

Turning to FIGS. 5-7, an alternate embodiment of a drainage valve 118 is illustrated. In this embodiment, the drainage valve 118 is a spring valve comprising a first valve connector element 124 and a second valve connector element 126. The first valve connector element 124 is in fluid communication with an interior of a drainage bag defining a closed urine collection chamber (not shown in FIG. 5), similar to the drainage bag 20 of FIGS. 1 and 1A.

A tapering mating portion 118a having a cylindrical opening 118d therein is provided on second valve connector element 126 so as to place the second valve connector element 126 in fluid communication with a second end of a tubing element (not shown in FIG. 5 or 6), similar to the second end of the tubing element 16b in FIG. 1 or 16b′ in FIG. 1A.

The second valve connector element 126 includes a valve plug 130 at a lower end thereof. The valve plug 130 is biased toward a closed condition by a valve spring 132 extending downwardly from a disc-like spring stage 133 having a round opening 135 therein, such that the valve plug 130 sealingly engages a valve seat 134 whenever the first valve connector element 124 is disengaged from the second valve connector element 126, thereby preventing the flow of urine through the drainage valve 118. Upon engagement of the first valve connector element 124 with the second valve connector element 126, an axial finger-like projection 136 provided on the first valve connector element 124 makes contact with a lowermost surface 130a of the valve plug 130 and urges the valve plug 130 against the biasing force of the valve spring 132 and off the valve seat 134, thereby placing a tubing element with which the second valve connector element 126 is engaged in fluid communication with a closed urine collection chamber or drainage bag 20.

The first valve connector element 124 and the second valve connector element 126 are selectively securable to one another. The first valve connector element 124 is provided with an annular wall 138 at an upper end thereof. This annular wall 138 includes a plurality of radially-outwardly projecting locking tangs 139. A plurality of locking fingers 140 project downwardly from an outer wall of the second valve connector element 126. Each of these locking fingers 140 includes an inwardly-directed locking tang 141 at a terminal end thereof that is complementary to a respective one of the radially-outwardly projecting locking tangs 139 of the first valve connector element 124. The locking fingers 140 can move axially along the annular wall 138 of the first valve connector element 124 in the gaps between the locking tangs 139, then rotate into locked engagement with the locking tangs 139 by rotating the first valve connector element 124 relative to the second valve connector element 126.

While there has been presented a detailed description for the purpose of understanding the present disclosure, it will be appreciated that the details herein given may be varied by those skilled in the art without departing from the true spirit and scope of the appended claims.

I claim:

1. A system for maintaining sterility in an indwelling catheter having a drain hub, comprising:
a tubing element having a first end connected to the drain hub of the catheter, the tubing element having a second end remote from the first end and the first and/or second ends including a drainage valve associated therewith, the drainage valve being movable from an open position for the drainage of urine from the catheter to a closed position for preventing the flow of urine from the catheter;
a drainage bag defining a closed urine collection chamber;
a first connector element associated with one of the drainage bag and tubing element and in fluid communication with the closed urine collection chamber;
a second connector element associated with the drainage valve for engagement with the first connector element, the first and second connector elements each including an internal engagement surface having an opening therein, the internal engagement surfaces being arranged for mating sealed engagement with the openings in alignment;
a retaining element for maintaining the internal engagement surfaces of the first and second connector elements in mating sealed engagement when the drainage valve is disposed in the open position to permit urine to flow through the catheter, through the tubing element, through the drainage valve, through the openings in the internal engagement surfaces of the first and second connector elements, and into the closed urine collection chamber of the drainage bag;
the retaining element being movable to a position allowing disengagement of the connector elements, the retaining element being movably coupled to the drainage valve so that movement of the retaining element for disengagement of the connector elements causes the drainage valve to move from the open to the closed position thereof, the drainage bag being removable from the system only when the drainage valve has been moved to the closed position; and
the internal engagement surface of the second connector element being exposed for contact with a sterilizing fluid when the first and second connector elements are not engaged.

2. The system of claim 1 wherein the drainage bag includes a removable region separate from the first connector element for creating a drainage spout for draining urine collected in the drainage bag from the closed urine collection chamber to thereby render it impossible for the drainage bag to be reused.

3. The system of claim 1 wherein before attachment of the drainage bag to the system a sterile barrier lid is peelably sealed to a perimeter surface of the first connector element to maintain sterility of the internal engagement surface of the first connector element and the inside of the drainage bag.

4. The system of claim 1 wherein the first end of the tubing element is a permanently connected to the drain hub of the catheter.

5. The system of claim 1 wherein the tubing element has a lumen extending from the first end to the drainage valve for carrying urine from the catheter.

6. The system of claim 1 wherein the drainage valve comprises a rotary valve adapted for rotary movement from the open position to the closed position.

7. The system of claim 6 wherein the opening in the second connector element is in alignment with a lumen of the tubing element.

8. The system of claim 7 wherein the rotary valve in the open position has a lumen in alignment with the opening in the second connector element.

9. The system of claim 7 wherein the rotary valve in the closed position has a lumen out of alignment with the opening in the second connector element.

10. The system of claim 7 wherein the rotary valve in the open position has a lumen in alignment with the lumen of the tubing element.

11. The system of claim 7 wherein the rotary valve in the closed position has a lumen out of alignment with the lumen of the tubing element.

12. The system of claim 6 wherein the retaining element is movable from engagement with, to disengagement from, the first connector element.

13. The system of claim 12 wherein the retaining element is in engagement with the first connector element in the open position of the drainage valve.

14. The system of claim 12 wherein the retaining element is disengaged from the first connector element in the closed position of the drainage valve.

15. The system of claim 12 wherein the retaining element has two slightly curved parallel fingers for cooperation with two slightly curved surfaces on opposite sides of the first connector element to maintain the first and second connector elements in mating sealed engagement during use.

16. The system of claim 7 wherein the first connector element is adapted for coaxially aligned telescopic engagement with the second connector element.

17. The system of claim 1 wherein the internal engagement surface of each of the first and second connector elements is a sloping surface, each of the first and second connector elements comprises a tubular segment, and the opening in each of the internal engagement surfaces is of reduced diameter relative to the corresponding tubular segment.

18. A system for maintaining sterility in a Foley catheter collection system, comprising:
a tubing element defining a catheter tube having an insertion end and an end remote from the insertion end, and including a drainage valve associated with the end remote from the insertion end of the catheter tube, the drainage valve being movable from an open position for draining urine through the catheter tube to a closed position for preventing the flow of urine from the catheter tube;
a drainage bag defining a closed urine collection chamber and a first connector element in fluid communication with the closed urine collection chamber;
a second connector element associated with the drainage valve for engagement with the first connector element, the first and second connector elements each including an internal engagement surface having an opening therein, the internal engagement surfaces being arranged for mating sealed engagement with the openings in alignment;
a retaining element for maintaining the internal engagement surfaces of the first and second connector elements in mating sealed engagement when the drainage valve is disposed in the open position to permit urine to flow through the catheter tube, through the drainage valve, through the openings in the internal engagement surfaces of the first and second connector elements, and finally to flow into the closed urine collection chamber of the drainage bag;
the retaining element being movable to a position allowing disengagement of the connector elements, the retaining element being movably coupled to the drainage valve so that movement of the retaining element for disengagement of the connector elements causes the drainage valve to move from the open to the closed position thereof, the drainage bag being removable from the system only when the drainage valve has been moved to the closed position; and
the internal engagement surface of the second connector element being exposed for contact with a sterilizing fluid when the first and second connector elements are not engaged.

19. The system of claim 18 wherein the drainage bag having a removable region separate from the first connector element for creating a drain spout for draining urine collected in the drainage bag from the closed urine collection chamber to thereby render it impossible for the drainage bag to be reused.

20. The system of claim 18 wherein before attachment of the drainage bag to the system a sterile barrier lid is peelably sealed to a perimeter surface of the first connector element to maintain sterility of the internal engagement surface of the first connector element and the inside of the drainage bag.

21. The system of claim 18 wherein the catheter tube has a lumen extending from the insertion end to the drainage valve for carrying urine from the bladder.

22. The system of claim 21 wherein the drainage valve comprises a rotary valve adapted for rotary movement from the open position to the closed position.

23. The system of claim 22 wherein the opening in the second connector element is in alignment with a lumen of the catheter tube.

24. The system of claim 23 wherein the rotary valve in the open position has a lumen in alignment with the opening in the second connector element.

25. The system of claim 23 wherein the rotary valve in the closed position has a lumen out of alignment with the opening in the second connector element.

26. The system of claim 23 wherein the rotary valve in the open position has a lumen in alignment with the lumen of the catheter tube.

27. The system of claim 23 wherein the rotary valve in the open position has a lumen out of alignment with the lumen of the catheter tube.

28. The system of claim 18 wherein the first and second connector elements are generally elliptically shaped for coaxially aligned telescopic engagement.

29. The system of claim 18 wherein the retaining element is movable from engagement with, to disengagement from, the first connector element.

30. The system of claim 29 wherein the retaining element is in engagement with the first connector element in the open position of the rotary valve.

31. The system of claim 29 wherein the retaining element is disengaged from the first connector element in the closed position of the rotary valve.

32. The system of claim 29 wherein the retaining element has two slightly curved parallel fingers for cooperation with two slightly curved surfaces on opposite sides of the first connector element to maintain the first and second connector elements in mating sealed engagement during use.

33. The system of claim 18 wherein the internal engagement surface of each of the first and second connector elements is a sloping surface, each of the first and second connector elements comprises a tubular segment, and the opening in each of the internal engagement surfaces is of reduced diameter relative to the corresponding tubular segment.

34. A system for maintaining sterility in an indwelling catheter having a drain hub, comprising:
a tubing element having a first end with a connector element for permanent connection to the drain hub of the catheter, the tubing element having a second end remote from the first end and including a drainage valve associated therewith, the drainage valve being movable from an open position for the drainage of urine from the catheter to a closed position for preventing the flow of urine from the catheter;
a drainage bag defining a closed urine collection chamber and a first connector element in fluid communication with the closed urine collection chamber;
a second connector element associated with the drainage valve for engagement with the first connector element, the first and second connector elements each including an internal engagement surface having an opening therein, the internal engagement surfaces being arranged for mating sealed engagement with the openings in alignment;
a retaining element for maintaining the internal engagement surfaces of the first and second connector elements in mating sealed engagement when the drainage valve is disposed in the open position to permit urine to flow through the catheter, through the tubing element, through the drainage valve, through the openings in the internal engagement surfaces of the first and second connector elements, and finally to flow into the closed urine collection chamber of the drainage bag;
the retaining element being movable to a position allowing disengagement of the connector elements, the retaining element being movably coupled to the drainage valve so that movement of the retaining element for disengagement of the connector elements causes the drainage valve to move from the open to the closed position thereof, the drainage bag being removable from the system only when the drainage valve has been moved to the closed position;
the retaining element having two slightly curved parallel fingers for cooperation with two slightly curved surfaces on opposite sides of the first connector element to maintain the first and second connector elements in mating sealed engagement during use;
the internal engagement surface of each of the first and second connector elements having a sloping surface, each of the first and second connector elements comprising a tubular segment, and the opening in each of the internal engagement surfaces being of reduced diameter relative to the corresponding tubular segment;
the internal engagement surface of the second connector element being exposed for contact with a sterilizing fluid when the first and second connector elements are not engaged;
the drainage bag including a removable region separate from the first connector element for creating a drainage spout for draining urine collected in the drainage bag from the closed urine collection chamber to thereby render it impossible for the drainage bag to be reused.

35. A system for maintaining sterility in a Foley catheter collection system, comprising:
a tubing element defining a catheter tube having an insertion end and an end remote from the insertion end, and including a drainage valve associated with the end remote from the insertion end of the catheter tube, the drainage valve being movable from an open position for draining urine through the catheter tube to a closed position for preventing the flow of urine from the catheter tube;
a drainage bag defining a closed urine collection chamber and a first connector element in fluid communication with the closed urine collection chamber;
a second connector element associated with the drainage valve for engagement with the first connector element, the first and second connector elements each including an internal engagement surface having an opening therein, the internal engagement surfaces being arranged for mating sealed engagement with the openings in alignment;
a retaining element for maintaining the internal engagement surfaces of the first and second connector elements in mating sealed engagement when the drainage valve is disposed in the open position to permit urine to flow through the catheter tube, through the drainage valve, through the openings in the internal engagement surfaces of the first and second connector elements, and finally to flow into the closed urine collection chamber of the drainage bag;
the retaining element being movable to a position allowing disengagement of the connector elements, the retaining element being movably coupled to the drainage valve so that movement of the retaining element for disengagement of the connector elements causes the drainage valve to move from the open to the closed position thereof, the drainage bag being removable from the system only when the drainage valve has been moved to the closed position;
the retaining element having two slightly curved parallel fingers for cooperation with two slightly curved surfaces on opposite sides of the first connector element to maintain the first and second connector elements in mating sealed engagement during use;
the internal engagement surface of each of the first and second connector elements having a sloping surface, each of the first and second connector elements comprising a tubular segment, and the opening in each of the internal engagement surfaces being of reduced diameter relative to the corresponding tubular segment;
the internal engagement surface of the second connector element being exposed for contact with a sterilizing fluid when the first and second connector elements are not engaged;
the drainage bag including a removable region separate from the first connector element for creating a drainage spout for draining urine collected in the drainage bag from the closed urine collection chamber to thereby render it impossible for the drainage bag to be reused.

36. A system for maintaining sterility in an indwelling catheter having a drain hub, comprising:
a tubing element having a first end connected to the drain hub of the catheter, the tubing element having a second end remote from the first end and the first and/or second ends including a drainage valve associated therewith, the drainage valve being movable from an open position for the drainage of urine from the catheter to a closed position for preventing the flow of urine from the catheter;
a drainage bag defining a closed urine collection chamber;
wherein the drainage valve comprises a spring valve comprising a first valve connector element and a second valve connector element selectively engageable with the first valve connector element, the second valve connector element including a valve spring therein that biases a valve plug against a valve seat and the first valve connector element includes an axial finger-like projection that, upon engagement of the first valve connector element with the second valve connector element, engages a surface of the valve plug and actuates the valve plug to a position off the valve seat; and
wherein the first valve connector element and the second valve connector element are each provided with a plurality of locking tangs, the plurality of locking tangs of one of the first and second valve connector elements including radially outwardly-projecting tangs and the plurality of locking tangs of the other of the first and second valve connector elements including inwardly-projecting tangs and being complementary to the outwardly-projecting locking tangs, the inwardly-projecting locking tangs, upon engagement with the outwardly-projecting locking tangs, securing the first and second valve connector elements to one another.

37. The system of claim 36, wherein the second valve connector element includes a tapering mating portion having a cylindrical opening therein so as to place the second valve connector element in fluid communication with the second end of the tubing element.

38. The system of claim 35, wherein the surface of the valve plug engaged by the axial finger-like projection upon engagement of the first valve connector element with the second valve connector element is a lowermost surface of the valve plug.

39. The system of claim 35, wherein the valve spring extends from a disc-like spring stage having an opening therein.
